(19) United States
(12) Patent Application Publication
(10) Pub. No.: US 2009/0239210 A1
(43) Pub. Date: Sep. 24, 2009

(54) Proteins recruited by virus during infection of plants and method for their isolation and identification

(76) Inventors: Christophe Brugidou, Pignan (FR); Jean-Paul Brizard, Saint Martin de Londres (FR)

Correspondence Address: NIXON & VANDERHYE, PC, 901 NORTH GLEBE ROAD, 11TH FLOOR, ARLINGTON, VA, 22203, US

(21) Appl. No.: 12/311,189

(22) PCT Filed: Sep. 24, 2007
(86) PCT No.: PCT/IB2007/003945

§ 371 Date: Mar. 20, 2009

Related U.S. Application Data
(60) Provisional application No. 60/847,085, filed on Sep. 26, 2006.

(51) Int. Cl. C12Q 1/00; G01N 33/00

(52) U.S. Cl. 435/4; 436/86

(57) ABSTRACT

The invention relates to a method for isolating and identifying proteins from protein-virus complexes and comprises: —the gel exclusion chromatography of virus-protein complexes extracted from infected plants or such as obtained by adding a purified virus to soluble proteins extracted from non infected plants, —SDS-PAGE of the proteins-analysis by NanoLC-MS/MS after tryptic digestion and protein identification.

The invention relates to proteins recruited by virus during infection of plants and a method for their isolation and identification.

To cope with the virus life cycle complexity (decapsidation-encapsidation, translation, replication, and transport) and host defenses, viruses are necessarily dependent on host proteins to carry out needed functions that are not encoded by the viral genome.

The recruiting of the translational machinery from host plants is now well established for different viruses, for instance in the case of Potyviruses, the interaction of eIF4E, eIFiso4E, eiF4G and the viral protein VPg.

Nevertheless, the question of how the virion acts in the cellular context remains.

Indeed, for each stage of the virus life cycle, it is necessary to have the required host partners in the right conditions of time, concentration, localization and conformation. It is also necessary for the virus to have a high affinity for some host proteins. The external part of virus particles could play this crucial function in recruiting host proteins.

To confirm this virus recruiting, the inventors developed a method using the rice yellow mottle sobemovirus (RYMV) as virus model, and the rice (Oryza saliva) as plant model which is fully sequenced and widely used for genomic studies.

RYMV causes substantial economic losses in rice production in Africa. Its distribution and biology are well known. This virus is a monopartite positive RNA strand virus about 4450 nucleotides, and its genome organization is quite simple as it is composed of four ORFs that encode at least five proteins. Three isoforms (compact, transitional and swollen) were identified according to the presence of divalent ions and pH, and were localized in different cell compartments such as cytoplasm, nucleus, vacuole, vesicle and cell wall. Viral particles were also suspected to be localized in chloroplast.

A range of analyses were developed by the inventors, from the isolation of virus-host protein complexes to the identification of host proteins by mass spectrometry, showing the specific recruiting of numerous host proteins by the virus.

It is thus an object of the invention to provide a method enabling isolation and identification of the host proteins recruited by a virus during an infection process.

It is another object of the invention to provide such proteins.

According to still another aspect, the invention relates to the use of such proteins as new therapeutic targets of great interest to prevent and/or treat host viral infections in plants.

The method of the invention for isolating and identifying proteins from protein-virus complexes, comprises:
the gel exclusion chromatography of virus-protein complexes extracted from infected plants or such as obtained by adding a purified virus to soluble proteins extracted from non infected plants,
SDS-PAGE of the proteins
analysis by NanoLC-MS/MS after tryptic digestion and protein identification.

The gel exclusion chromatography comprises injecting, in a chromatography column, a supernatant such as obtained by centrifugation of a suspension of crushed frozen leaves in an extraction buffer at pH 7.5, and is followed by the recovery and lyophilisation of the fractions containing the virus-protein complexes.

To perform said SDS-PAGE, the proteins are denatured by heating after adding a SDS loading buffer and a gel is loaded.

After in gel digestion, the proteins are extracted and injected for NanoLC-MS/MS. The mass data recorded and the proteins are identified by comparing with protein databases.

More particularly, the major functional category of the proteins isolated according to the above method category correspond to proteins involved in metabolism functions, mainly glycolysis, photosynthesis, amino acid, lipid and cell-wall metabolism.

The second category correspond to functions involved in translation and protein synthesis (T) including translation factors, elongation factors, tRNA synthetases, protein disulfide isomerase, chaperone proteins, and proteasome.

The third category is related to defense (D), with protein chaperones (i.e. 70, 82 and 90 kDa), proteins involved in defense pathways such as superoxide dismutase (SOD), phenylalanine-ammonia lyase (PAL), homocystein S-methyltransferase, lipoxygenase, proteins related to oxidative stress with thioredoxin, peroxiredoxin, oxidoreductase NAD binding, glutathion-S transferase as well as pathogenesis related proteins including peroxidase and chitinases.

As shown in the examples, said proteins may be recruited by different viruses particularly the same proteins from the same plant may be recruited by unrelated viruses.

Other categories, less represented, were related to unknown functions (U), transport (Tp) and transcription (Tr).

The invention particularly relates to a method such as above defined wherein the virus is rice Yellow Mottle Virus (RYMC) or FHV.

This new molecular methods could also be applied to human and animal viruses for the purpose of finding new therapeutic targets.

This experimental approach is also useful to in vivo and in vitro isolate virus/host proteins complexes to find new therapeutic targets in human and animal virus diseases. Advantageously, this method is based on the separation of the complexes under their native form by exclusion chromatography.

The invention also relates to the use of said method to identify new target proteins belonging to said complexes to find new markers for plant selection, or to develop new strategies to abort virus infection processes. Said identification may be carried out by usual methods of mass spectrometry after separation on SDS-PAGE gel.

The method of the invention is also useful to identify partners directed bound to the virus, by carrying out a separation step using a salt gradient. The extractions of the viral complexes may be carried out at salt concentrations of 0.5 m and 1 M which abolish the low intensity interactions and consequently enable to only maintain in the proteic complexes the proteins having a strong interactions between them. Interestingly, the above disclosed method allows comparisons between proteomes of virus complexes (sensible, resistant . . . ), the identification of involved proteins and paralogs to identify factors of sensitivity, resistance. The extraction of viral complexes obtained from plants having different sensibilities with respect to the virus allows the identification of proteins which are involved in the interactions and which are specific of the sensibilities or the resistance to said plants.

By using said method, it is also possible to analyse virus-proteins reconstitutions made with non host proteins, which may indicate weaknesses in the host barrier. The viral complexes obtained, in vitro, according to the invention, with proteins of non host plants allows the identification of proteins capable of promoting the passing round of the host barrier.

The method of the invention is useful to generate new targets for resistance strategies. The proteins identified in the viral complexes are surexpressed or silenced in order to develop a resistance to the infection by a virus.

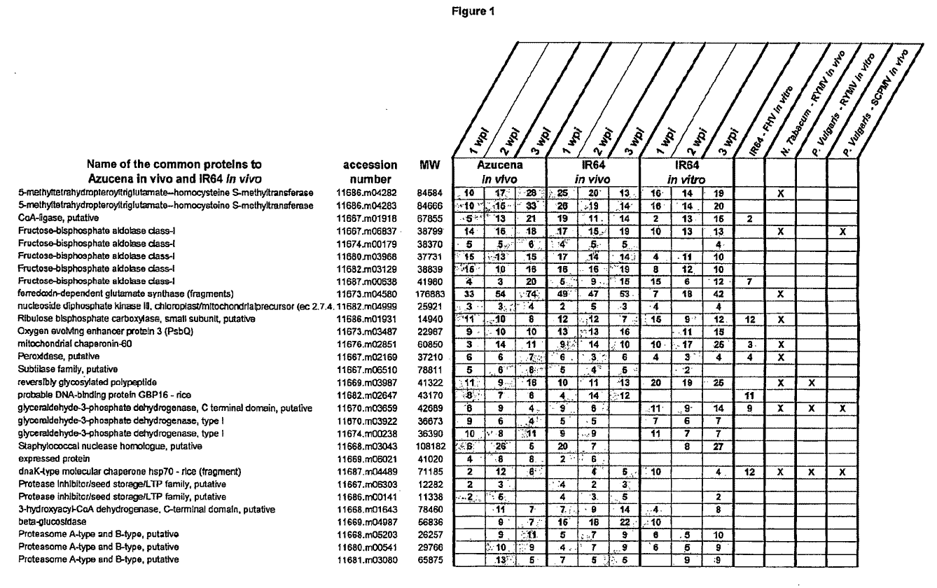

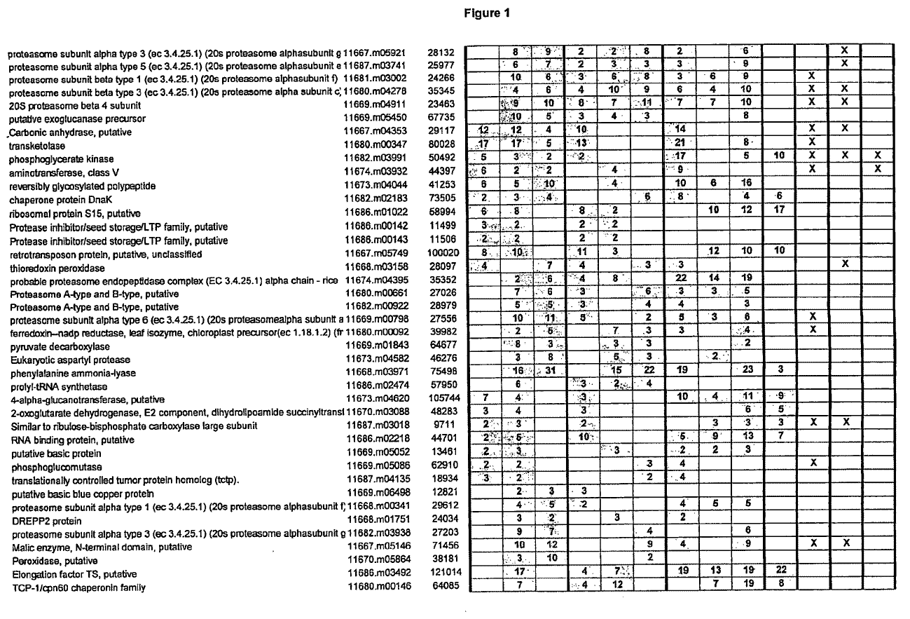

Other characteristics and advantages of the invention are given in the following examples which refer to FIGS. 1 to 8, wherein

FIG. 1: gives the name of the common proteins to Azucena in vivo and IR64 in vivo, the specific proteins to Azucena in vivo and to IR64 in vivo/in vitro,

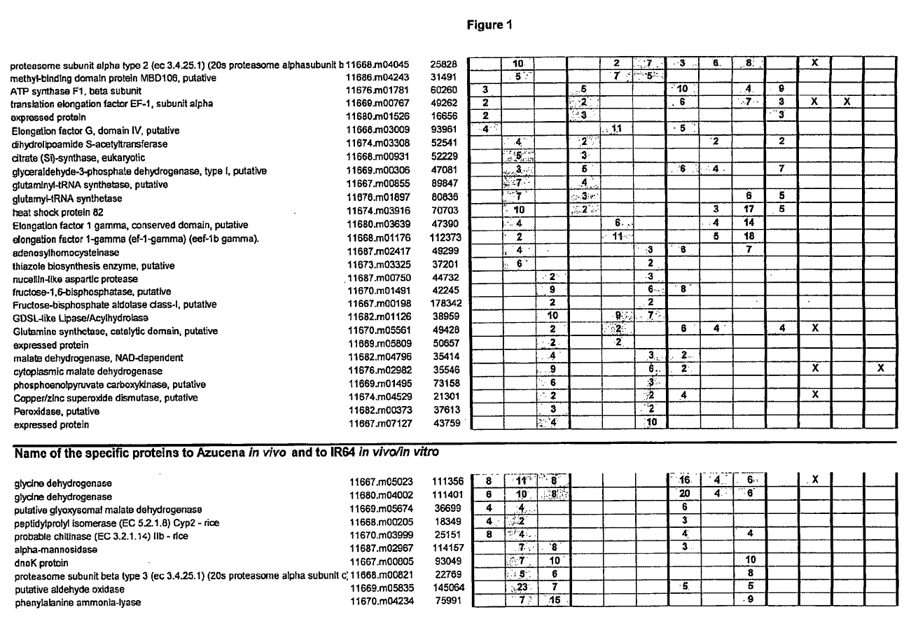

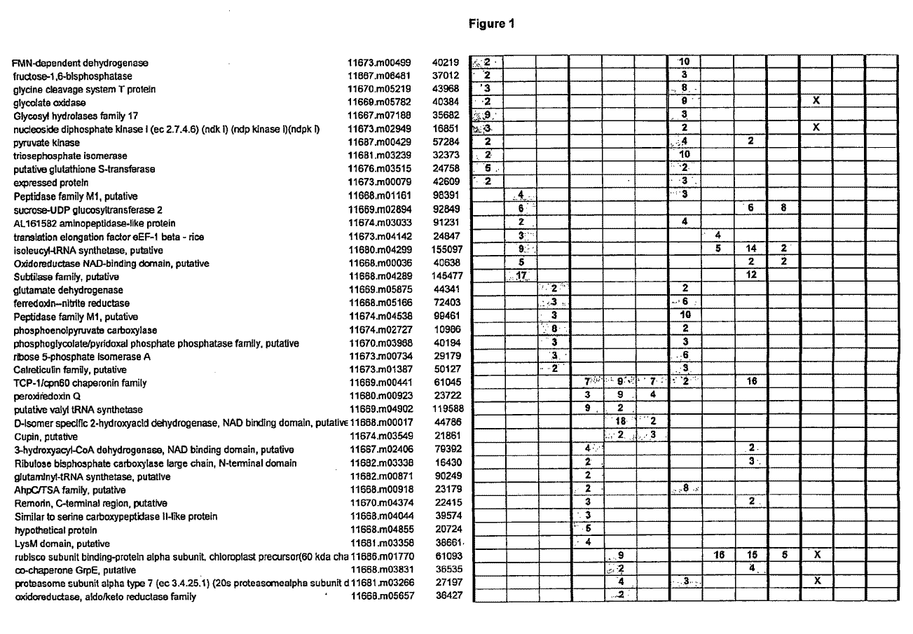

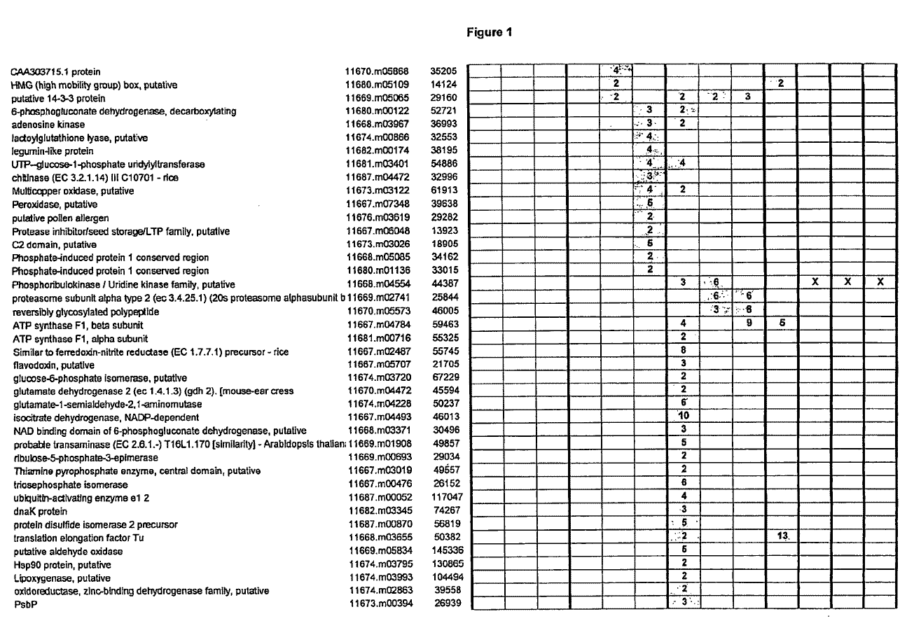

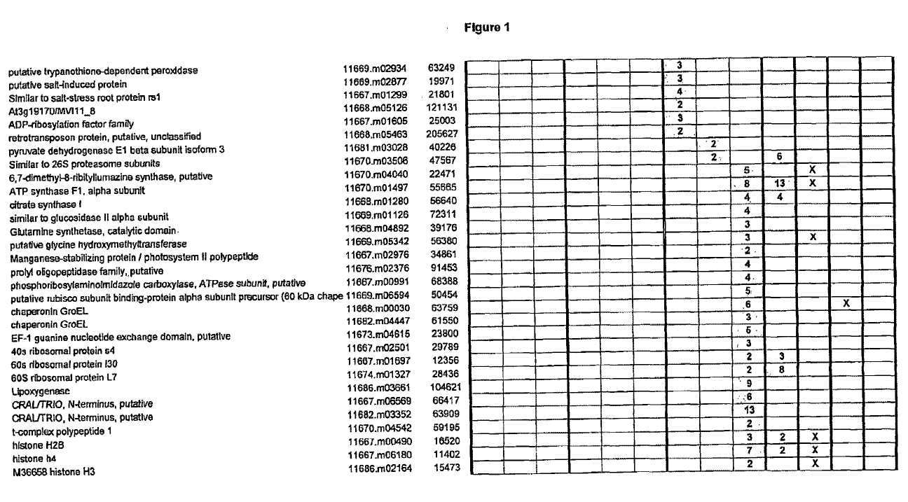

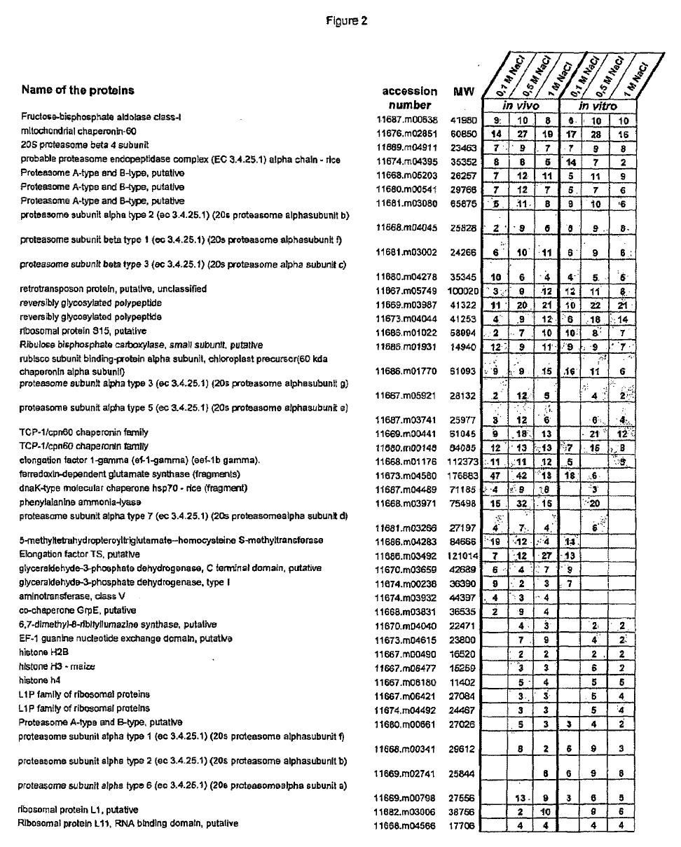

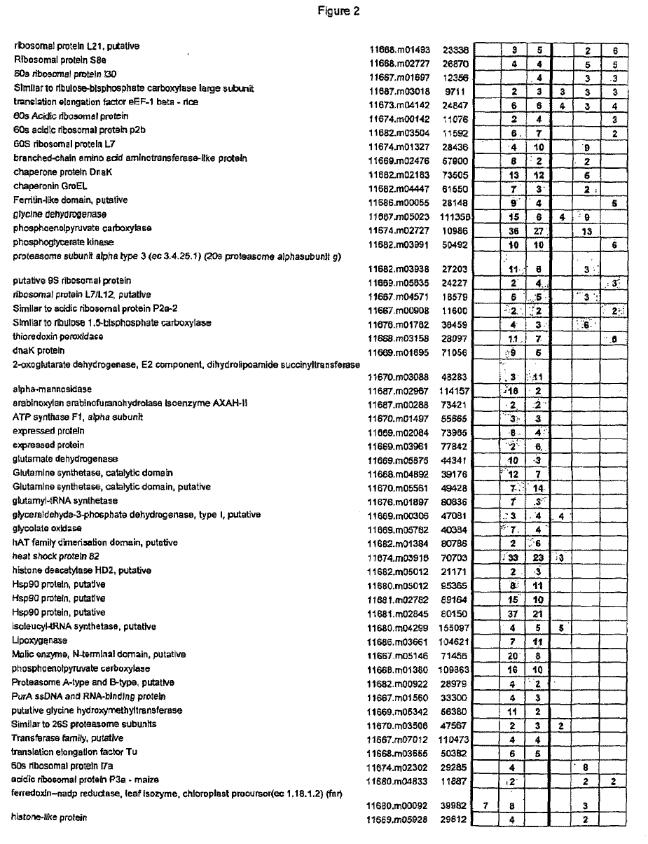

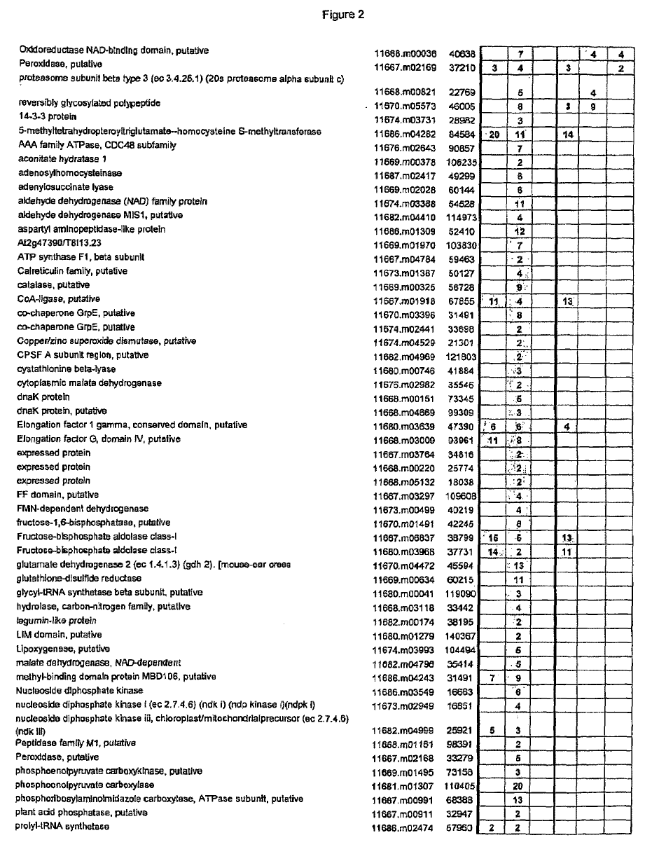

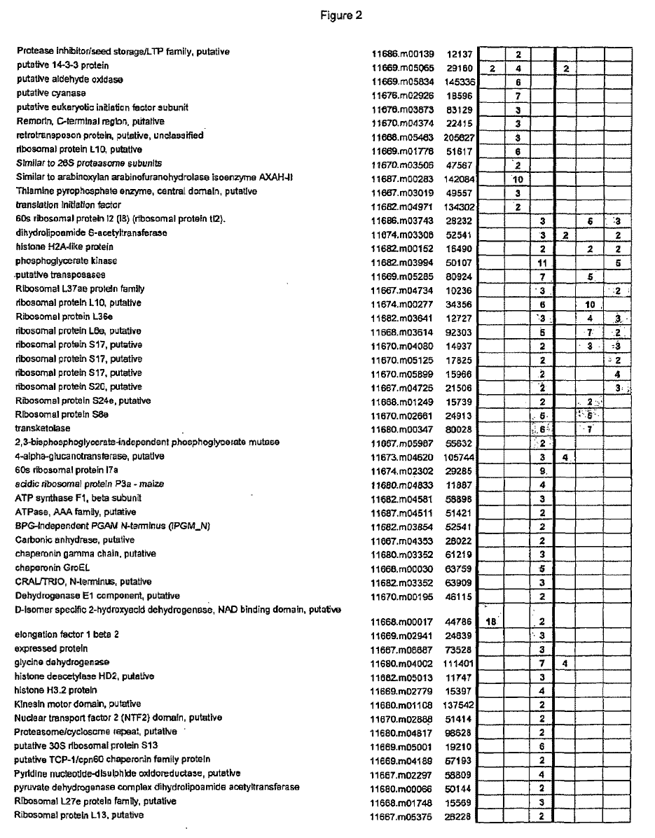

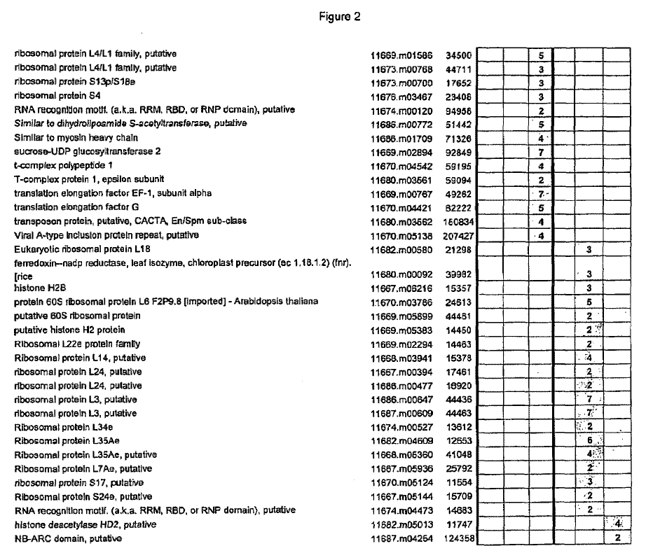

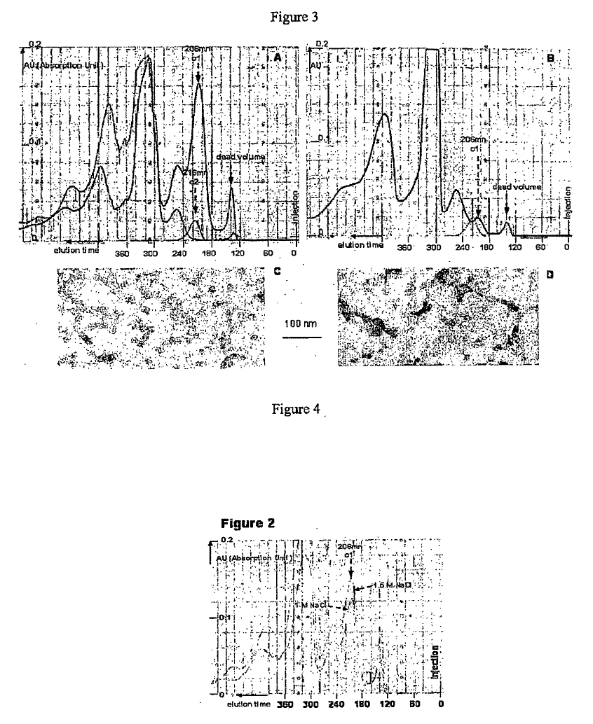

FIG. 2: the name of the proteins identified from in vivo and in vitro complexes at different salt concentration,

FIG. 3: the elution profiles of IR64 protein extraction solutions from in vivo and in vitro binding experiments, at using 0.1 M NaCl,

FIG. 4: the elution profiles of IR64 protein extraction solutions from in vivo using 1 M or 1.5 M NaCl

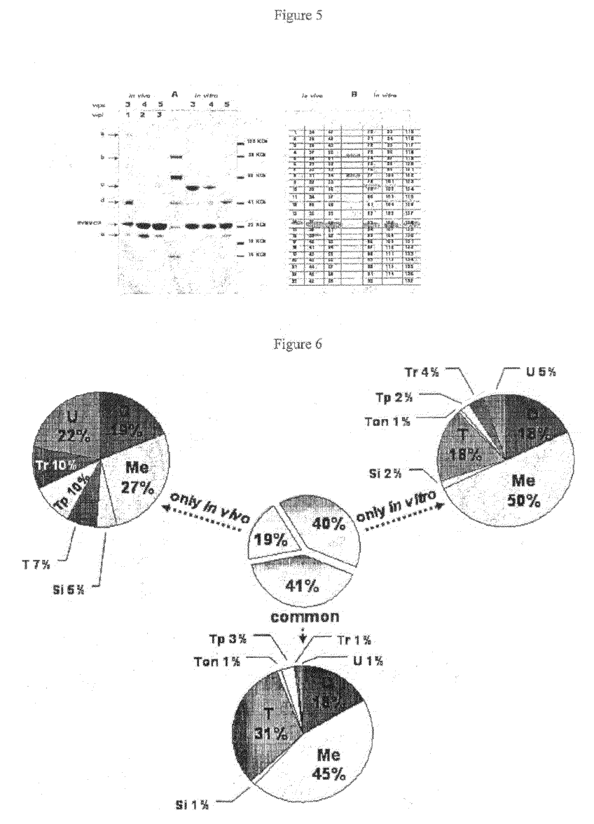

FIG. 5: the gels of denatured RYMV-IR64 protein complexes,

FIG. 6: the distribution of proteins identified from RYMV-IR64 experiments in Table 1 of FIG. 1,

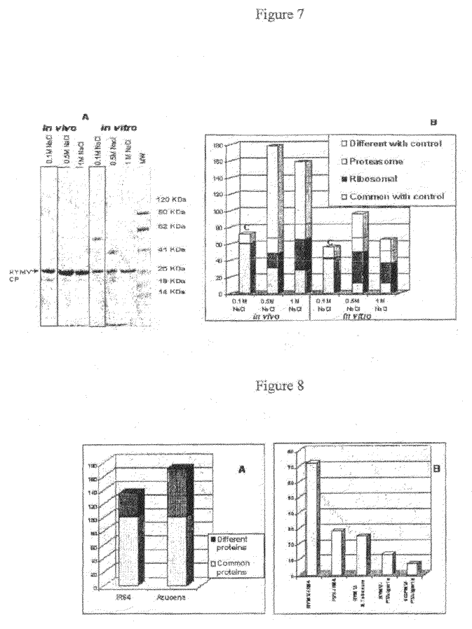

FIG. 7: the effects of increased NaCl concentration during extraction-purification of complexes,

FIG. 8: the common protein from different complexes.

### EXPERIMENTAL PROCEDURES

### Plant and Virus Materials

Two rice cultivars showing a contrasted response to RYMV infection were used: a highly susceptible one IR64 (Oryza sativa indica), and a partially resistant and tolerant one Azucena (Oryza sativa japonica). IR64 is a high-yielding cultivar developed at the International Rice Research Institute (IRRI) and Azucena is a traditional upland cultivar from the Philippines.

For both cultivars, seeds were sown separately and plants were grown in a confined greenhouse in controlled conditions: 12 h light at 28° C. and 12 h dark at 24° C. Two weeks after seedling, plants were mechanically inoculated with purified RYMV particles from a virulent isolate of Burkina Faso (BF1) at a concentration of 100 μg/ml in inoculation buffer (20 mM phosphate, pH 7) as described previously (1). This experiment was repeated twice. Leaves of non-inoculated and RYMV inoculated plants were harvested at 1, 2 and 3 weeks post inoculation (wpi), frozen in nitrogen and conserved at −80° C.

To compare the specificity of the recruited host proteins, Phaseolus vulgaris (non host for RYMV and host for Southern cowpea mosaic virus, SCPMV) and Nicotiana tabacum (non host for RYMV) were used.

Infected and/or non-infected plants were cultivated and harvested in the same conditions as rice cultivars.

Different viruses were used in the experiments disclosed hereinafter: RYMV isolate BF1 (access. Q9DGX1 Swiss Prot Database) was used to infect rice cultivars, for in vitro binding experiments with rice host plant and with N. tabacum as non host plant.

SCPMV belongs to the same genus Sobeinovirus, but has a different genomic organisation and a limited dicotyledonous host range.

Flock house virus (FHV) is a member of the insect and animal virus family Nodaviridae.

### RYMV Extraction

Rice leaves were ground in liquid nitrogen and homogenized in 0.1M phosphate buffer, pH 5.0. Virus particles were precipitated with 6% PEG 8000 and resuspended in phosphate buffer. Further purification was performed by centrifugation through a 10 to 40% sucrose gradient. Virus concentration was estimated by spectrophotometry using an extinction coefficient of 6.5.

### Plant Protein Extraction

About 10 g of frozen leaves were crushed in a liquid nitrogencooled mortar, and 50 ml of buffer extraction pH7.5 (Tris-HCl 50 mM, NaCl 100 mM, EDTA 10 mM, Glucose 25 mM, EGTA 5 mM, DTT 5 mM, Glycerol 5%, 0.1% Triton X100, Protease Inhibitor Cocktail Tablets (Roche Applied Science) added to the resulting powder.

The suspension was centrifuged at 22000 g for 30 min at 3° C. and the supernatant was filtered through a 0.45 μm syringe filter.

### Extraction of Virus-Protein Complexes

Frozen infected leaves (1, 2, 3 wpi) were treated with the protocol used for plant protein extraction (see below), then concentrated to 10 ml by ultra filtration with Centricon Plus 20 (Millipore) at 3° C.

### In vitro Virus-Proteins Binding Assay

3.5 mg of purified virus were added to 100 mg proteins extracted with buffer as described above, then concentrated to 10 ml by ultra filtration with Centricon Plus 20 at 3° C. Contact between virus and proteins were approximately 90 min until injection on chromatographic column. All purification steps were done in a cold room at 4° C.

### Purification of Virus-Protein Complexes

The concentrated mixtures were injected on a 1000 mm long/26 mm diameter column filled with Sephacryl S500 (Amersham Biosciences), and TAPS buffer pH 7.6, 100 mM NaCl as eluent. The AKTAprime system (Amersham) was used for injection, detection and collection of the proteins. Fractions corresponding to the virus-proteins complexes were collected and lyophilized (c1, FIG. 1).

### SDS-PAGE of Virus-Proteins Complexes

The lyophilized pellets were resuspended with deionized water (Milli-Q) and the resulting solution was desalted using PD10 columns (Amersham Biosciences). The amount of protein was estimated using the BCA Protein Assay Kit (Pierce) and about 40 μg of proteins were denatured for 5 nm at 100° C. after adding of 2×SDS-loading buffer (100 mM Tris-HCl pH6.8, 200 mM DTT, 4% SDS, 0.2% Bromophenol Blue, 20% Glycerol) and loaded on pre-cast gel 4-12% acrylamide (Invitrogen).

Gels were stained with colloidal Coommassie blue, and sample bands were obtained from gels under sterile conditions to avoid keratin contamination (FIG. 2).

### Mass Spectrometry and Protein Identification

In-gel digestion was performed with an automated protein digestion system, MassPREP Station (Waters, Milford Mass., USA). The gel plugs were washed three times with a mixture of 50%/50% NH4HCO3 (25 mM)/ACN. The cysteine residues were reduced with dithiothreitol at 57° C. and alkylated with iodoacetamide. After dehydration with acetonitrile, the proteins were digested in-gel with 30 μL of 12.5 ng/mL of modified porcine trypsin (Promega, Madison, Wis., USA) in 25 mM NH4HCO3 overnight and at room temperature.

Then a double extraction was performed, first with 60% acetonitrile in 5% formic acid and a second one with 100% acetonitrile. The resulting peptide extracts were directly injected for nanoscale capillary liquid chromatography-tandem mass spectrometry (nanoLC-MS/MS) analysis.

NanoLC-MS/MS analysis of the digested proteins was performed using a CapLC capillary LC system (Waters, USA) coupled to a hybrid quadrupole orthogonal acceleration time-offlight tandem mass spectrometer (Q-TOF II, Waters). Chromatographic separations were conducted on a reverse-phase (RP) capillary column (Pepmap C18, 75 μm i.d., 15 cm length, LC Packings) with a 200 mL/min flow rate.

Mass data acquisitions were piloted by MassLynx software (Waters, USA) using automatic switching between MS and MS-MS modes as described [2,3]. To improve the quality of MS/MS spectra during nanoLC-MS/MS analysis,

energy curves depending on the m/z value of the selected precursor ion were empirically derived: for each m/z value, 3 different collision energies were applied. Fragmentation was performed using argon as the collision gas.

The mass data recorded during nanoLC-MS/MS analysis were processed and converted into MassLynx .pkl peak lists format prior to searching with the search engine Mascot (Matrix Science, London, UK). The searches were performed on a local Mascot server running on a 3 GHz Pentium IV processor with a tolerance on mass measurements of 70 ppm in MS mode and 0.25 Da in MS/MS mode. One missed cleavage per peptide was allowed and some variable modifications were taken into account such as carbamidomethylation for cysteine, oxidation for methionine and N-acetylation of the protein. Searches were performed without constraining protein molecular weight or isoelectric point. The Rice (Nippon Bare) pseudomolecule database (release 3) accessible from the TIGR (The Institute for Genomic Research http://www.tigr.org/tdb/e2k1/osa1/) website was used for the identification of rice proteins and a compilation database gathering SwissProt, TrEMBL and TrEMBLnew protein databases for the identifications of the other host plant proteins (Phaseolus vulgaris and Nicotiana tabacum).

A total of 531 proteins were annotated for Phaseolus vulgaris and 1988 proteins for Nicotiana tabacum in this compilation database.

The rice protein identification method using the complete pseudomolecule database, allowed, in some cases, to discriminate paralog genes among multigenic families.

Due to the strategy developed here, the virus coat protein was present in all gel bands and in very high concentration independently of the spot position on the gel. To circumvent the superabundance of the viral coat protein, an informatics exclusion program was used to prevent the selection of the coat protein peptides during the nanoLC-MS/MS analysis. This procedure allowed the identification of lower abundant proteins.

A python language script was used to extract data from files generated by Mascot software, and the Access Data Base software (Microsoft) was used to gather and compare datasets according to the different conditions of the experiments.

In this study, an identification was validated when the protein was identified by at least two peptides both having an MS/MS ion score higher than 39.

### Results

### Methodology Used to Isolate RYMV Host Protein Complexes In Vivo and In Vitro

RYMV-host protein complexes were isolated from infected plants or from in vitro binding experiments with soluble proteins extracted from non infected leaves supplemented with purified virus.

10 ml of concentrated protein extract solution are injected with an automated injection syringe (ATKTA Prime system), and elution was carried out at 1.5 ml/mn with buffer elution (TAPS 20 mM pH 7.6, 100 mM NaCl). Dead volume represents the fraction of membrane fragments and/or protein aggregates that are still present in solution.

The results are given on FIG. 3A-D wherein collected fractions containing RYMV-host proteins complexes correspond to the first ⅔ part of peaks labeled c1.

(A) Size exclusion chromatography elution profiles of purified RYMV (red), of soluble proteins extracted from IR64 non infected leaves (Control experiment in green) and IR64 infected leaves (blue).

(B) Size exclusion chromatography of in vitro binding experiment with soluble proteins extracted from IR64 healthy plants added with purified RYMV.

(C) Negative contrast electron micrograph of collected fraction stained with uranyl acetate containing purified RYMV particle from fraction corresponding to the peak c1 eluted at 216 nm.

(D) RYMV particle associated with host materials in fraction corresponding to the peak c2 eluted at 206 mm. The bar represents 100 nm.

According to the used protocol (see Experimental Procedures), after injection of purified virus (6.5 mg) an elution peak was detected at 216 minutes. The first elution peak of soluble proteins extracted from non infected Oryza sativa (IR64) leaves, which was the control without virus (negative control), was eluted at 255 minutes (FIG. 1A, green) and overlapped slightly with the peak at 216 minutes corresponding to the experiment with the virus purified alone (FIG. 1A, c2, red). The eluted profile of soluble proteins extracted from infected leaves showed an additional peak at 206 minutes corresponding to virus-protein complexes (FIG. 1A, c1 blue).

The same additional peak at 206 minutes was found in experiments using the buffer extraction added with 0.5M or 1M NaCl (FIG. 4). In experiments with in vitro virus-host protein complexes, the same additional peak at 206 minutes was detected at a lower concentration in accordance with the amount of added purified virus (3.5 mg) (FIG. 3B, c1).

After the purification/elution cycle, electron microscopy revealed the presence of intact virus particles in the collected fraction corresponding to peak c2 (FIG. 3C). In collected fraction corresponding to peak c1, virus particles were associated with electron dense materials (FIG. 3D). This method thus allowed to isolate in vivo and in vitro virus-host protein complexes and was further used to identify virus-host protein partners by nanoLC-MS/MS.

### SDS-PAGE Separation

At different times of plant development (3, 4 and 5 weeks post seedling, wps) and during the time course of infection (1, 2 and 3 weeks post infection, wpi) virus-host protein complexes were isolated from in vivo and in vitro experiments, denatured and separated by SDS-PAGE.

The results are given on FIGS. 5A and 5B: 5A: SDS-page gel electrophoresis of RYMV-IR64 protein complexes collected after size exclusion chromatography from in vitro IR64 infected leaves, and from in vitro binding experiments. Plants at 1, 2 and 3 weeks post infection (wpi) correspond to plants at 3, 4 and 5 weeks post seedling (wps); 5B: Same gel showing the numbering and localization of samples analyzed by LC-MS-MS. Each gel lane was cut and analyzed after tryptic digestion.

Gel protein profiles were reproducible when virus, plant varieties and stage of development were identical. On the contrary the protein profiles for O. sativa IR64 variety differed, according to the stage of development and the time course of infection for in vivo experiments or stage of development for in vitro experiments.

When comparing the same stage of development, specific profiles were observed for in vivo and in vitro experiments. This observation supports the idea that complexes isolated from infected plants were already present in the in vivo tissues, and were not formed during the protein extraction process. Thus, for visible bands as a, b, c, d and e, different accumulations were observed between in vivo and in vitro experiments at different stages of development. To identify these proteins, systematic cutting and nanoLC-MS/MS analysis of these gel lanes were performed.

### Identification of Virus Host Protein Partners by Mass Spectrometry

The RYMV coat protein (CP, accession number: Q9DGX1) was identified around 26 kDa in accordance with the results obtained by Western blot using monoclonal antibody against the virus. For the same total amount (20 μg) of proteins loaded on each lane, the amount of CP increased during the time course of infection, whereas, equal amounts were observed for complexes purified from in vitro experiments (FIG. 5A).

The virus coat protein was present at such a high concentration that it was identified in all gel bands independently from location in the gel. As the nanoLC-MS/MS automatic acquisition program selected the most prevalent peptides for fragmentation, the high abundance of the coat protein prevented the identification of less abundant proteins.

To circumvent this problem, an exclusion program was established to informatically exclude the masses of the coat protein tryptic peptides, thereby preventing their selection and allowing the selection of peptides belonging to other proteins.

As shown in FIG. 5B, 137 gel bands were analyzed by nanoLC-MS/MS for the experiments with the IR64 cultivar with RYMV and 2017 proteins were identified in the rice pseudomolecules release 3 database.

Nevertheless, most of the proteins were identified several times and presented only the 223 non-redundant identified proteins are presented in Table 1 of FIG. 1.

### Functional Distribution of Recruited Host Proteins

For the 223 identified proteins using the IR64 cultivar, the following distribution was found: 19% were identified only in vivo, 41% were identified both in vivo and vitro, and 40% were present only in vitro.

The results are illustrated by FIG. 6 wherein the central circle is divided in proteins detected only in vivo experimentations, only in vitro experimentations, and detected in vivo as well in vitro experimentations (common).

The following mentions are given for each group functional distribution: Me (metabolism), D (defense-stress), Tr (transcription), T (translationlprotein synthesis), Tp (transport), Si (signal transduction), U (unknown), To (transposon).

Proteins identified only in vivo experimentations (19%) might be explained by the specificity of a recruiting made in planta, and by the inability for these already coated virus particles to recruit more proteins during the extraction process.

Proteins identified only in vitro experimentations (40%) might be explained by a recruiting due to the solubilization of proteins that were not accessible for the virus in planta.

Some of these proteins were identified in vivo experimentations with Azucena cultivar (i.e. putative aldehyde oxidase 11669.m05835) and might be involved in a specific recruiting in planta, but for the other we could not exclude an artifact recruiting. For the common proteins, it was highly probable that they could be recruited in planta, and they were recruited in vitro because they are extracted in vitro experimentations.

### Paralogs Identification

As shown with the paralog 11668.m03971 for phenylalanine ammonia-lyase (Defense category). Said paralog was indeed it is possible to discriminate which paralog gene among a multigenic family was a specifically recruited and present in virus-host protein complex specifically identified among 10 paralogs (TIGR database) from this multigenic family.

In addition, some specific paralogs were recruited only in vivo such as peroxidase, putative 11682.m00373, others in vitro such as reversibly glycosylated polypeptide 11670.m05573.

Additionally, the same paralogs identified in vivo and in vitro (peroxidase, putative 11667.m02169 for example) were observed. The number of recruited paralogs may vary with the experimental conditions as shown with (Glyceraldehyde-3-phosphate dehydrogenase in Metabolism category) 4 paralogs (Table 1) were recruited in vivo and in vitro by the virus at one week post infection, 3 paralogs were still recruited in vivo at 2 wpi instead of 4 in vitro, and finally no paralog from G3PDH was recruited in vivo at 3 wpi whereas 3 paralogs were identified in vitro at 3 wpi.

Specific paralogs among multigenic families may then be recruited at specific points during the time course of virus infection.

### Specificity of Host Proteins Recruiting

According to the invention order to investigate the specificity of host proteins recruiting by RYMV, the following strategy was used:
(i) different NaCl concentrations were used to extract and isolate complexes from in vivo or in vitro experiments in order to confirm that some of the protein interactions occurred at low ionic strength, and may have been unspecific;
(ii) It was then investigated whether the recruited proteins were host and virus dependent by testing different virus-host pairs.

Varying NaCl concentration: The effects of increased NaCl concentration during extraction purification of complexes were studied. The results are given on FIGS. 7A and 7B; 7A: SDS-page gel electrophoresis of RYMV-host protein complexes extracted and purified by size exclusion chromatography from 2 wpi in vivo IR64 infected leaves or from in vitro binding experiments at different salt concentrations. Extractions and exclusion size chromatography were realized with buffers added with 0.1 M NaCl, 0.5M NaCl then 1M NaCl.; 7B: Histogram of non-redundant proteins identified by LC-MS-MS (see Table 2) in comparison with a control C (IR64 2 wpi 0.1M NaCl in vivo, and IR64 4 wps 0.1M NaCl in vitro). Ribosomal proteins and histon proteins (except ribosomal protein S1 5, putative 11686.m01022) were identified only at 0.5 M and 1M NaCl.

Protein profiles were quite different according to the salt concentration used in vivo and in in vitro extractions (FIG. 7A). As expected, the amount of CP from in vitro experiments at different salt concentrations was similar. On the contrary, in in vivo experiments a higher amount of CP was observed at 0.5 M and 1M NaCl, suggesting that a higher amount of complexes was extracted with a high ionic strength buffer, probably due to the better extraction of some subcellular compartments.

The higher number of proteins identified at 0.5 M and 1M of NaCl confirmed this result, specifically the ribosomal proteins which were localized in nucleus, mitochondria and chloroplasts and also histon proteins which were localized in nucleus (in vivo and in vitro, FIG. 7B), and furthermore, proteins from proteasome which localized to cytoplasm are well identified at low as well as high ionic strength (as cytoplasmic compartment is easier to extract) (Table 2 on FIG. 2). An expected decrease of total proteins at 1 M of NaCl (151 vs 163 at 0.5 M for in vivo and 62 vs 93 at 0.5M for in vitro) was obesrved. The common proteins that were still identified at 1M NaCl suggest that the binding was specific and that they strongly interacted with virus particles (Table 2).

Two effects of salt concentration were shown by said results:
first, an increase of salt concentration to 0.5 M increased the number of proteins bound to the virus, resulting from a better extraction of complexes;
on the contrary, the increase of salt concentration to 1 M reduced the number of common proteins that were also identified at 0.1M NaCl, suggesting that proteins still identified at high salt concentration were strongly specific of the complexes.

### Different Pairs of Virus-Host Plant

To see further whether this affinity was host and virus dependent, complexes extracted and purified from various virus-hosts combinations were studied.

The results are given on FIGS. 8A and 8B which gives: 8A:the histogram of cumulated number of non-redundant proteins identified by LC-MS-MS (see Table 1) for IR64 and Azucena cultivars infected by RYMV at 1, 2 and 3 wpi; 8B: the histogram of common proteins identified in FHV-IR64 in vitro interaction (see Table 1) and common protein functions identified in different interactions (in vitro with RYMV-N. tabacum and RYMV-P. vulgaris; in vivo with SCPMV and P. vulgaris)* (see Table 1). * Identification was made using the TIGR rice pseudomolecule for experimentations with IR64 and Azucena cultivar, and with SwissProt, TrEMBL and TrEMBLnew for Phaseolus vulgaris and Nicotiana tabacum.

First, the in vivo interaction of RYMV with two sub-species of O. sativa were compared at 1, 2 and 3 wpi the susceptible O. sativa ssp indica (IR64) and the tolerant O satliva ssp japonica (Azucena) (FIG. 8A).

More proteins were identified for Azucena than for IR64 (171 vs 135 proteins identified when 1, 2 and 3 wpi identifications were cumulated).

Among these proteins, a large number (100) were common for both sub-species (Table 1), confirming that the recruiting among sub-species was quite similar. It was likely that the proteins identified differentially in Azucena and IR64 take part to the tolerance or susceptibility of these subspecies (FIG. 3).

The recruiting between different viruses and different host-plants was further investigated: (i) a compatible interaction between another sobemovirus (SCPMV) and his host plant Phaseolus vulgaris (ii) three incompatible interactions, one between an insect virus FHV and IR64, and two other pairs: RYMV-N. tabacum and RYMV-P. vulgaris. Some identical protein functions were identified among all the different complexes (FIG. 7B).

For the interaction between FHV and IR64, 41 common proteins were identified and also found with the interaction RYMV-IR64 (Table 1), what could explain the ability of FHV to replicate in rice, and show that a set of proteins from a host plant interact with different viruses.

The results obtained with RYMV, FHV and SCPMV suggested that host protein recruiting occurred for different viruses and that two unrelated viruses (RYMV and FHV) could recruit the same proteins from the same plant.

Thus, using size exclusion chromatography, virus-protein complexes were purified from infected plants, and from in vitro binding experiments using purified virus and soluble proteins extracted from non-infected plants. This method is reproducible and allowed to purify enough material to analyze complexes by SDS-PAGE and nanoLC-MS/MS.

Host proteins from the complexes were separated and gave reproducible protein profiles for the same experimental conditions.

To demonstrate the specific recruiting by the virus, three critical stages for RYMV were studied: 1 wpi (beginning of replication), 2 wpi (replication in systemically infected leaves, first symptoms) and 3 wpi (end of viral replication in susceptible variety IR64 and development of symptoms). It was then observed that the recruiting of host proteins were different according to the infection stages.

Comparing the same infection stage in vitro and in vivo, the inventors demonstrated that the complexes isolated from infected plants were not formed during the extraction, but, preexisted in vivo during the infection process.

The number of identified proteins for each stage (1, 2 and 3 wpi) corresponds to a population of different complexes representative of the global situation within infected plants, as the virus has the ability to infect different cell compartments. Looking at the stained SDS-PAGE gels, some recruited proteins show different quantitative profiles according to the different stages of infection (band a, b, c, d and e, FIG. 4). Because of the reproducibility of this recruiting, these results reinforce the idea that virus recruits specific host proteins during the infection process.

Among the recruited proteins, some of them have a higher affinity for RYMV. This was supported by the identification of 32 proteins that were still binding to the virus at 1 M of NaCl in vivo (out of 72 at 0.1M NaCl for IR64 2 wpi), and among them, 25 proteins were identified in vitro at 1M of NaCl (FIG. 5B).

These proteins are most likely bound directly at the surface of the virus particle.

The other proteins that were identified in lower salt concentration could have a lower affinity with the surface of the virus or could bind host proteins previously recruited by the virus.

Interestingly, a recruiting coherence was found through some functional categories.

In metabolism category a high number of enzymes involved in glycolysis, malate and citrate cycles, were identified presumably recruited by the virus to produce energy for virus replication.

In the defense category, proteins involved in ROS (reactive oxygen species) and detoxification (superoxide radical and hydrogen peroxide) were identified which are presumably recruited by the virus to maintain an oxido-reduction environment compatible with viral replication.

In addition, in the protein synthesis category, a set of proteins involved in translation processes with ribosome, elongation factors, protein chaperones, protein disulfide isomerase and proteins involved in protein turn-over with the proteasome 20S were identified.

All these proteins would be recruited by the virus to optimize virus translation efficiency, as soon as virus starts decapsidation.

Virus recruiting is likely to occur for other viruses, as we saw a recruiting for the couple SCPMV-Phaseolus vulgaris (FIG. 8B). RYMV is able to recruit proteins from Nicotiana Tabacum and from Phaseolus vulgaris in in vitro experimentations (FIG. 8B).

The results obtained in vitro with the pair FHV-IR64 allow the identification of 41 proteins that where also found with the pair RYMV-IR64 (in vivo and/or in vitro), showing then a common set of proteins recruited from rice by viruses able to replicate in this plant. It was demonstrated that the FHV is able to replicate and is systemically spread in rice plants expressing movement protein genes.

Inside the rice host sub-species studied, it was also shown that most recruited proteins are identical (FIG. 8A), the proteins recruited that are different between the two sub-species could be related to the susceptibility or tolerance effect observed for the IR64 and Azucena varieties.

Some proteins are identified whatever the experimental conditions (i.e. mitochondrial chaperonin60 11676.m02851), suggesting that they have a very strong affinity for viruses and that they play an important role in the virus biological process.

Some paralog genes belonging to a multigenic family that were specifically recruited were discriminated.

Some of the proteins identified in the complexes from the IR64-RYMV and Azucena-RYMV experiments were shown as deregulated in IR64 and Azucena suspension cells undergoing RYMV infection. About fifteen similar protein functions were found in other study using cDNA-AFLP to discover genes induced or repressed during virus infection.

Some of the proteins have been identified in different virus-host interactions such as Hsp 60 with Hepatitis B virus or HIV, Hsp70 with plant closteroviruses or HIV what suggests that viruses may recruit the same protein functions in different hosts.

Other proteins, non identified in said experiments, were identified interacting with different viruses, as pectin methylesterases, homeodomain proteins, rab acceptor-related proteins, β-1,3-glucanase-interacting proteins, Fas-mediated apoptosis enhancer Daxx, SUMO-1 protein.

All these results demonstrate that the recruiting of proteins appears to be a common process for different viruses.

### REFERENCES

1. N'Guessan, P., Pinel, A., Caruana, M. L., Frutos, R., Sy, A. A. et al. (2000) Evidence of the presence of two serotypes of rice yellow mottle sobemovirus in Côte d'Ivoire. Eur J Plant Pathol 106, 167-178
2. Ventelon-Debout, M., Delalande, F., Brizard, J. P., Diemer, H., Van Dorsselaer, A. et al. (2004) Proteome analysis of cultivar-specific deregulations of Oryza sativa indica
3. Richert, S., Luche, S., Chevallet, M., Van Dorsselaer, A., Leize-Wagner, E. (2004)

1. A method for isolating and identifying proteins from protein-virus complexes, comprises: the gel exclusion chromatography of virus-protein complexes extracted from infected plants or such as obtained by adding a purified virus to soluble proteins extracted from non infected plants, SDS-PAGE of the proteins—analysis by NanoLC-MS/MS after tryptic digestion and protein identification.

2. The method according to claim 1 wherein the gel exclusion chromatography comprises injecting in a chromatography column, a supernatant such as obtained by centrifugation of a suspension of crushed frozen leaves in an extraction buffer at pH 7.5, and is followed by the recovery and lyophilisation of the fractions with the virus-protein complexes.

3. The method of claim 1, wherein the proteins are denatured by heating after adding a SDS loading buffer and a gel is loaded to perform said SDP-PAGE.

4. The method of claim 1, comprising in gel digestion, extracting the protein and injecting for NanoLC-MS/MS, recording the mass data and identifying by comparing with protein databases.

5. The method of claim 4, further comprising extracting the data using software to gather and compare data sets according to different conditions of the experiments.

6. The method according to claim 1, wherein the isolated proteins of the proteins isolated according to the above method category correspond to proteins involved in metabolism functions, mainly glycolysis, photosynthesis, amino acid, lipid and cell-wall metabolism.

7. The method according to claim 1, wherein discrimination of paralog genes belonging to a multigenic family specifically recruited.

8. The method according to claim 1, wherein the isolated proteins functions involved in translation and protein synthesis (T) including translation factors, elongation factors, tRNA synthetases, protein disulfide isomerase, chaperone proteins, and proteasome.

9. The method according to claim 1, wherein the isolated proteins related to defense (D), with protein chaperones (i.e. 70, 82 and 90 kDa), proteins involved in defense pathways such as superoxide dismutase (SOD), phenylalanine-ammonia lyase (PAL), homocystein S-methy transferase, lipoxygenase, proteins related to oxidative stress with thioredoxin, peroxiredoxin, oxidoreductase NAD binding, glutathion-S transferase as well as pathogenesis related proteins including peroxidase and chitinases.
